(19) United States
(12) Patent Application Publication
(10) Pub. No.: US 2009/0117658 A1
(43) Pub. Date: May 7, 2009

(54) Macrophage transfection method

(75) Inventors: Thomas E. Wagner, Greenville, SC (US); Gunter Schwamberger, Salzburg (AT); Xianzhong Yu, Mauldin, SC (US)

Correspondence Address: FOLEY AND LARDNER LLP;SUITE 500, 3000 K STREET NW, WASHINGTON, DC, 20007, US

(73) Assignee: Greenville Hospital System

(21) Appl. No.: 12/149,097

(22) Filed: Apr. 25, 2008

Related U.S. Application Data
(60) Provisional application No. 60/907,977, filed on Apr. 25, 2007.
(60) Provisional application No. 60/924,868, filed on Jun. 4, 2007.

(51) Int. Cl. C12N 15/86; C12N 15/06; C12N 15/64

(52) U.S. Cl. 435/456; 435/455; 435/320.1

(57) ABSTRACT

Described are a method and a composition for transfecting monocytes, as well as use of the same for therapeutic purposes. The composition is composed of a nucleic acid component, a lysosome evading component and a digestible particle that can be phagocytized. Preferably, the monocyte is a macrophage and the digestible particle is from a natural source, such as from a microbial source. More preferably, the digestible particle is a yeast cell wall particle such as zymosan. The composition itself, or cells pretreated with the composition, are useful in all gene medicine applications, such as gene therapy, gene vaccination, cancer treatment as well as immunomodulation and tissue repair.

### CROSS-REFERENCE TO RELATED PATENT APPLICATIONS

This application claims priority from U.S. Provisional Application 60/907,977, filed Apr. 25, 2007 and U.S. Provisional Application 60/924,868, filed Jun. 4, 2007, incorporated herein by reference in their entirety.

### FIELD OF THE INVENTION

The present invention relates to a method for transfecting a monocyte and therapeutic uses thereof.

### BACKGROUND OF THE INVENTION

Monocytic cells play a central role in the immune response. They mature into macrophages and dendritic cells, i.e., the major antigen presenting cells of the body. Moreover, as tumors grow, they produce macrophage attracting chemokines, as part of the angiogenic process, which draw monocytic cells to the tumor. Thus, monocytic cells, if specifically targeted, could be used to either deliver therapeutic gene products to tumor cells or generate a therapeutic or prophylactic immune response via their superior antigen presenting properties.

Monocyte-derived cells normally patrol the body in search of foreign, non-self structures, typically microbes. The monocytic cells phagocytose the microbes, which are then digested to smaller antigenic portions in the lysosome. The resultant antigens are cycled back to the surface for presentation to the humoral and cellular arms of the immune system.

Macrophages are cells within the tissues that originate from monocytic cells. They are of particular interest because they play an important role in both nonspecific and specific defenses in the host against pathogens. Recently, it has been found that macrophages also have a supportive, rebuilding function in the host. For example, Schwartz et al. (Journal of Neurotrauma, 23: 360-370, 2006) demonstrate that local implantation of activated macrophages promotes functional recovery of damaged spinal cord. It is believed that the initial microglial response to central nerve system (CNS) injury is beyond the capacity of the CNS to tolerate it. Therefore, immune-based intervention, such as by local injection of activated macrophages, proves to minimize neurological damage after acute spinal cord injury (SCI).

U.S. Pat. No. 6,875,612 to Wagner et al., which is specifically incorporated by reference, describes directed entry of a bead vector into monocytic cells for delivery of therapeutic gene products to tumor cells. The '612 patent, however, does not describe the use of a yeast cell wall particle such as zymosan to direct entry of a nucleic acid into a cell of monocytic origin.

Zymosan is an insoluble polysaccharide component of yeast cell wall. Prior publications uncovered zymosan's involvements in (i) induction of the release of cytokines or proinflammatory cytokines, (ii) induction of protein phosphorylation and inositol phosphate formation, (iii) arachidonate mobilization, (iv) activation of the alternative complement pathway; and (v) raise of cyclin D2 levels, suggesting a role of cyclin D2 in macrophage activation (Miyasato et al., Int. Arch. Allergy Immunol. 104: 24-26, 1994). For example, it has been reported that zymosan particles are capable of inducing inflammatory signals in macrophages through Toll-like receptors, e.g. TLR2 and TLR6, and dectin-1, which is a receptor that binds β-glucans and is important for macrophage phagocytosis. Zymosan is also involved in inducing inflammatory responses, such as TNF-α production and NF-κB activation in macrophages (Underhill, Journal of Endotoxin Research, 9: 176-180, 2003; Sato et al., J. Immunol., 171: 417-425, 2003; Dillon et al., J. Clin. Invest. 116: 916-928, 2006).

The present inventors have surprisingly discovered that macrophages can better tolerate phagocytosing a composition comprising a nucleic acid component attached to a digestible particle, such as a yeast cell wall particle, than a composition comprising an undigestible particle or a particle made from non-natural sources, such as ferromagnetic beads. Thus, the present invention is an improvement over prior art strategies using macrophages for gene delivery. “Better tolerance” can be quantified by various assays detecting metabolism and viability. For example, macrophage survival and vitality after ingestion of particulate vectors can be monitored with tetrazolium dye (MTT staining), and trypan blue exclusion assays assess cell death.

### SUMMARY OF THE INVENTION

In the first aspect, the present invention provides a composition for directed entry into a monocyte. The composition comprises (i) a nucleic acid component, (ii) a lysosome evading component and (iii) a digestible particle that can be phagocytosed. In one embodiment, the nucleic acid component contains a non-replicative and/or non-infective, form of a virus, which contains nucleic acid that encodes a protein. The non-replicative and/or non-infective form of a virus can act as a lysosome evading component and therefore, a second additional lysosome evading component is optional.

In some embodiments, the nucleic acid component may be DNA or RNA. In one embodiment, the nucleic acid may encode a protein such as an antigen or other therapeutic protein or a RNAi construct. In another embodiment, the nucleic acid component comprises a nucleic acid encoded in an expression vector containing a nuclear promoter, such as CMV promoter or a hypoxia induced promoter. In yet another embodiment, the nucleic acid may be encoded in a cytoplasmic vector, such as a T7 vector system.

In other embodiments, the lysosome evading component can be a virus or a component of a virus, such as an adenovirus or the adenovirus penton protein.

In certain embodiments, the particle that can be phagocytosed is one that is digestible and approximates the size of the microbial structures that monocytic cells typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to +/−0.25 μm. Preferably, the particle is from a natural source, such as a microbial particulate structure. For example, the particle that can be a phagocytosed is yeast cell wall particle, such as zymosan, or a beta glucan or a peptidoglycan from gram positive bacteria. Other suitable particles that can be phagocytosed, however, include agarose and insulin.

In some embodiments, the composition may further contain a nucleic acid protecting component, such as protamine, polyarginine, polylysine, histone, histone-like proteins, synthetic polycationic polymers or core protein of a retrovirus with the appropriate packaging sequence included in the RNA sequence.

The components may be attached to the digestible particle by any means which allows for attachment. In one embodiment, the nucleic acid and the lysosome evading component are attached to the particle by antibody attachment. In another embodiment, the nucleic acid and the lysosome evading component are attached to the particle by interaction between (strept)avidin and biotin. In yet another embodiment, the nucleic acid serves as a multiple binding vehicle.

In a related aspect, the invention provides a method for transfecting monocytes. The method comprises contacting a monocytic cell, such as a dendritic cell or a macrophage, with the above-described composition.

In another aspect, the invention provides a method for targeted delivery of a biological material into a monocytic cell by contacting the monocyte, such as a dendritic cell or a macrophage, with the above-described composition.

In yet another aspect, the present invention provides a gene therapy method involving administering the above-described composition to a person in need thereof, wherein the nucleic acid component comprises a nucleic acid that encodes a therapeutic protein, such as an anti-tumor protein. The present invention provides a method for gene vaccination involving administering the above-described composition to a person in need thereof, wherein the nucleic acid encodes an antigen, such as allergens, viral antigens, bacterial antigens or antigens derived from parasites. The present invention also provides a method for cancer treatment involving administering the above-described composition to a person in need thereof, wherein the nucleic acid is an anti-tumor gene, such as an anti-angiogenic factor, an immunomodulator or an anti-inflammatory factor.

In a further aspect, the present invention provides a method for tissue repair in general, such as spinal cord repair, comprising administering the above-described composition to a person in need thereof. The present invention also provides for a method for immunomodulation. For example, treating chronic inflammatory diseases like rheumatoid arthritis by direct injection into the joint, and other forms of autoimmune inflammation are also contemplated by the present invention.

In the above described methods, the composition may be administered intravenously or subcutaneously. For example, the monocytic cells may be transfected with the particle-conjugated virus (e.g., zymosan-conjugated virus) ex vivo and then reinfused into the patient intravenously. This mode of administration may be most suitable for targeting tumors. Alternatively, the particle-conjugated virus may be administered by applying locally, either by direct injection or microsurgery techniques. This method of administration may be most suitable for spinal cord repair or treating rheumatoid arthritis. One of skill in the art would know which mode of administration would be suitable for treating a given condition.

### BRIEF DESCRIPTION OF THE DRAWINGS

FIG. 1 is an adenovirus vector containing siRNA for an Iκβ gene fused to green fluorescence protein (GFP).

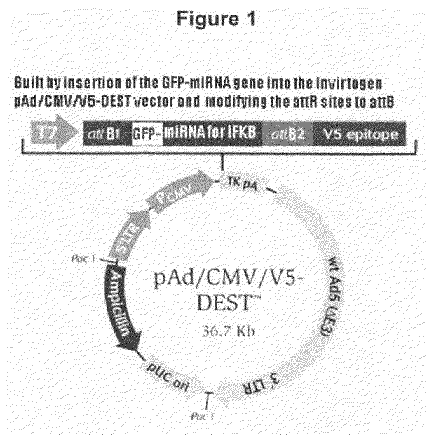

FIG. 2 is a photograph of macrophages that have engulfed GFP-RNAi constructs for IκB (MB-GFP-RNAi and Z-GFP-RNAi). (A) streptavidin coated magnetic beads (“MB”) attached to a biotinylated GFP-RNAi adenovirus vector or (B) zymosan (“Z”) particles attached to the GFP-RNAi adenovirus vector.

FIG. 3 depicts the cytotoxic activity of culture media of macrophages transfected with zymosan-conjugated Ad vectors. The culture medium of these cells was collected after 48 h, concentrated and tested for antitumor activity. These data demonstrate successful transfection and activation of macrophages by the zymosan-conjugated siRNA vectors.

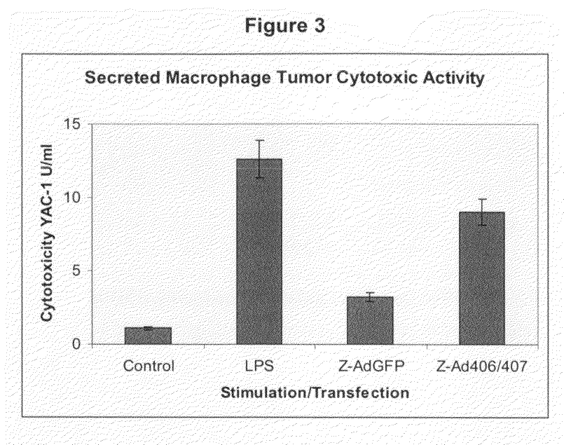

### DETAILED DESCRIPTION OF THE INVENTION

### Introduction

The present invention provides a composition for transfecting a monocytic cell, as and related methods of use. The composition for transfecting a monocytic cell according to the present invention is generally composed of a nucleic acid component, attached to a digestible particle that can be phagocytosed and a lysosome evading component. In one embodiment, the digestable particle that can be phagocytosed is a particle from natural sources, preferably of microbial origin, and most preferably a yeast cell wall particle. The composition of the present invention is also referred to herein as a “digestible particle containing composition.”

In one embodiment, the nucleic acid component may also be the lysosome evading component. The inventive composition attracts cells of monocytic origin, such as dendritic cells and macrophages, which renders the composition extremely useful for gene medicine methods, such as gene vaccination, gene therapy and cancer treatment.

Monocytes are phagocytic immune cells that ingest particulate structures such as microbes. The present invention takes advantage of this property in that the vectors are provided on a substrate that “looks” like a microbe.

### Digestible Particle

A preferred size for the digestible particle is one that approximates the size of microbial structures that monocytic cells typically ingest. In one embodiment, the particle will be about 0.05 to about 5.0 μm, about 0.05 to about 2.5 μm, about 0.1 to about 2.5 μm, about 1.0 to about 2.5 μm, about 1.0 to about 2.0 μm, or about 1.0 to about 1.5 μm. The term “about” in this context refers to +/−0.25 μm. Preferably, the digestible particle is a particle from natural sources, such as a particle of microbial origin. A particularly preferred particle is a yeast cell wall particle. In one embodiment, the yeast cell wall particle is a zymosan particle. Zymosan (also referred to as Zymosan A) is commercially available from various companies such as. Sigma-Aldrich. The zymosan particle size is typically about 2.0 μm.

On the other hand, for manufacturing purposes, slightly larger particles are preferred, because they are less likely to stick together, and so washing free from bound components is easier with the larger particle sizes.

The digestible particle is not limited by shape or material. In general, the particle can be of any shape, size or material that allows the digestible particle containing composition to be phagocytized by monocytes, such as dendritic cells and macrophages.

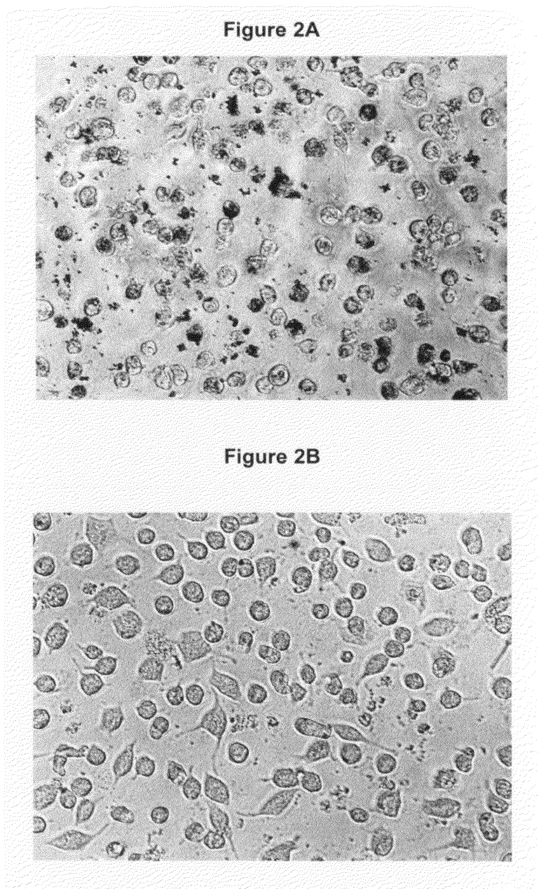

Surprisingly, the inventors discovered that a phagocytosed digestible particle appears to be better tolerated by the macrophages than a phagocytosed synthetic bead. In fact, as demonstrated in FIG. 2, the macrophages that engulfed zymosan particles attached to AD-GFP vector have a more natural appearance than the macrophages that engulfed a streptavidin-coated magnetic bead attached to a biotinylated AD-GFP vector.

### Nucleic Acid Component

The digestible particle of the present invention generally is attached to a nucleic acid component. The nucleic acid component comprises a nucleic acid that encodes a protein or an RNAi construct, and can be composed of DNA, RNA or both DNA and RNA. The nucleic acid component can also comprise a vector which contains the nucleic acid. The component typically contains the signals necessary for translation and/or transcription (i.e., it can ultimately encode a protein or an RNA product).

The artisan immediately will comprehend the large number of proteins that can be encoded by the nucleic acid. Typically, they will be antigens or anti-tumor proteins such as anti-angiogenic proteins and interleukins. The proteins will be localized predominantly in the immediate vicinity of a tumor via the macrophage.

Exemplary antigens useful in vaccine applications include allergens, viral antigens, bacterial antigens and antigens derived from parasites. Preferred antigens include tumor associated antigens, with which the artisan will be familiar (e.g., carcinoembryonic antigen, prostate-specific membrane antigen, melanoma antigen, adenocarcinoma antigen, leukemia antigen, lymphoma antigen, sarcoma antigen, MAGE-1, MAGE-2, MART-1, Melan-A, p53, gp 100, antigen associated with colonic carcinoma, antigen associated with breast carcinoma, Muc1, Trp-2, telomerase, PSA and antigen associated with renal carcinoma. Viral antigens also are preferred. Suitable viral antigens include HIV, EBV and Herpesvirus. In one embodiment, the nucleic acid encodes a linear gp41 epitope insertion (LLELDKWASL), which has been identified as a useful construct for improving HIV-1 Env immunogenicity (Liang, et al., Vaccine, 16; 17(22):2862-72, July 1999).

As provided above, the nucleic acid is preferably encoded in an expression vector which is capable of expressing the protein products of the nucleic acid. The vector typically further comprises regulatory sequences, including for example, a promoter, operably linked to the coding sequence. The vector may further comprise a selectable marker sequence, for instance for propagation in in vitro bacterial or cell culture systems. Preferred expression vectors comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 or cytomegalovirus (CMV) viral genome, for example, SV40 origin, early promoter, enhancer, splice, and polyadenylation sites may be used to provide the required nontranscribed genetic elements.

Specific initiation signals may also be required for efficient translation of inserted target gene coding sequences. These signals include the ATG initiation codon and adjacent sequences. In cases where a nucleic acid component includes its own initiation codon and adjacent sequences are inserted into the appropriate expression vector, no additional translation control signals may be needed. However, in cases where only a portion of an open reading frame (ORF) is used, exogenous translational control signals, including, perhaps, the ATG initiation codon, must be provided. Furthermore, the initiation codon must be in phase with the reading frame of the desired coding sequence to ensure translation of the entire target.

These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:516-544 (1987)). Some appropriate expression vectors are described by Sambrook, et al., in Molecular Cloning: A Laboratory Manual, Second Edition, Cold Spring Harbor, N.Y. (1989), the disclosure of which is hereby incorporated by reference. If desired, to enhance expression and facilitate proper protein folding, the codon context and codon pairing of the sequence may be optimized, as explained by Hatfield et al., U.S. Pat. No. 5,082,767.

Promoters include CMV immediate early, HSV thymidine kinase, early and late SV40, LTRs from retrovirus, and mouse metallothionein-I. Preferred promoters are those that are target specific, i.e., promoters that permit expression of a particular gene in a specific area targeted for treatment. For example, a suitable promoter for use in the present invention when targeting a tumor cell would be a hypoxia induced promoter.

Exemplary vectors include pWLneo, pSV2cat, pOG44, pXT1, pSG (Stratagene) pSVK3, pBPV, pMSG, and pSVL (Pharmacia). Selectable markers include CAT (chloramphenicol transferase). Preferred vectors also include cytoplasmic vectors, like the T7 vector system. See Wagner et al., U.S. Pat. No. 5,591,601 (Jan. 7, 1997).

### Lysosome Evading Component

In addition to the nucleic acid component, the yeast cell wall particle also generally has attached to it a lysosome evading component. The role of the lysosome evading component with is to assist the nucleic acid component in escaping the harsh environment of the lysosome. Aside from those disclosed herein, the skilled artisan will be aware of numerous examples of such molecules.

When a monocytic cell ingests a large antigen, a phagocytic vesicle (phagasome) which engulfs the antigen is formed. Next, a specialized lysosome contained in the monocyte fuses with the newly formed phagosome. Upon fusion, the phagocytized large antigen is exposed to several highly reactive molecules as well as a concentrated mixture of lysosomal hydrolases. These highly reactive molecules and lysosomal hydrolases digest the contents of the phagosome. Therefore, by attaching a lysosome evading component to the particle, the nucleic acid that is also attached to the particle escapes digestion by the materials in the lysosome and enters the cytoplasm of the monocyte intact. Prior systems failed to recognize the importance of this feature and, thus, obtained much lower levels of expression than the present invention. See Falo et al., WO 97/11605 (1997). It should be noted that the term “lysosome evading component” encompasses the fused lysosome/phagosome described above.

The lysosome evading component is any component that is capable of evading or disrupting the lysosome. For example, the lysosome evading component can include proteins, carbohydrates, lipids, fatty acids, biomimetic polymers, microorganisms and combinations thereof. It is noted that the term “protein” encompasses a polymeric molecule comprising any number of amino acids. Therefore, a person of ordinary skill in the art would know that “protein” encompasses a peptide, which is understood generally to be a “short” protein. Preferred lysosome evading components include proteins, viruses or parts of viruses. The adenovirus penton protein, for example, is a well known complex that enables the virus to evade/disrupt the lysosome/phagosome. Thus, either the intact adenovirus or the isolated penton protein, or a portion thereof (see, for example, Bal et al., Eur J Biochem 267:6074-81 (2000)), can be utilized as the lysosome evading component. Fusogenic peptides derived from N-terminal sequences of the influenza virus hemagglutinin subunit HA-2 may also be used as the lysosome evading component (Wagner, et al., Proc. Natl. Acad. Sci. USA, 89:7934-7938, 1992).

Other preferred lysosome evading components include biomimetic polymers such as Poly (2-propyl acrylic acid) (PPAAc), which has been shown to enhance cell transfection efficiency due to enhancement of the endosomal release of a conjugate containing a plasmid of interest (see Lackey et al., Abstracts of Scientific Presentations: The Third Annual Meeting of the American Society of Gene Therapy, Abstract No. 33, May 31, 2000-Jun. 4, 2000, Denver, Colo.). Examples of other lysosome evading components envisioned by the present invention are discussed by Stayton, et al. J. Control Release, 1; 65(1-2):203-20, 2000.

### Nucleic Acid Protection Component

In addition to the components described above which are generally attached to the digestible particle, either directly or via attachment to one another (e.g., a recombinant adenovirus encoding a nucleic acid), other components may also be attached to the particle or to a component that is attached to the particle. For example, a DNA protecting component may optionally be added to the digestible particle containing compositions described above, especially where the nucleic acid component is not associated with a virus or a portion thereof. Generally, the DNA protecting component will not be attached directly to the digestible particle. The nucleic acid protecting component includes any component that can protect the digestible particle-bound DNA or RNA from digestion during brief exposure to lytic enzymes prior to or during lysosome disruption. Preferred nucleic acid protecting components include protamine, polyarginine, polylysine, histone, histone-like proteins, synthetic polycationic polymers and core protein of a retrovirus with the appropriate packaging sequence included in the RNA sequence.

In one embodiment of the present invention, the digestible particle containing composition comprises (i) a recombinant, optionally non-replicative and/or non-infective, form of a virus, which contains a nucleic acid encoding a protein, and (ii) a digestible particle that can be phagocytized. The virus may be an RNA virus, like a retrovirus, or a DNA virus, like an adenovirus. In this embodiment, the virus itself preferably is capable of lysosome disruption. In other words, the nucleic acid and lysosome evading components are both integral parts of the virus. Alternatively, the virus may not be capable of lysosome disruption. In such a case, of course, a separate lysosome evading component should be added. Preferred viruses include HIV, adenovirus, Sindbis virus, and hybrid and recombinant versions thereof. A particularly preferred virus is an HIV-adenovirus hybrid, which is essentially a recombinant adenovirus that has been engineered to express HIV antigens. Viruses can be attached to the digestible particles directly, using conventional methods. See Hammond et al., Virology 254:37-49 (1999).

Since viral infection is not essential in the present invention for the nucleotide component to reach the cytoplasm of the monocyte, the virus can also be replication/infection deficient. One method for producing a replication/infection deficient adenovirus envisioned by the instant invention is altering the virus fiber protein. For example, a virus in which the fiber protein is engineered by specific mutations to allow the fiber protein to bind to an antibody but not to its cognate cellular receptor can be used in the instant invention.

Another method for producing a replication/infection deficient virus envisioned by the present invention is intentionally causing denaturation of the viral component responsible for infectivity. In the case of adenovirus, for example, the fiber protein could be disrupted during the preparation of the virus; for HIV it might be the envelope (env) protein. A method for producing a replication/infection deficient retrovirus envisioned by the present invention entails removing the outer membranes of the retrovirus so that only the retrovirus core particle remains. If a replication/infection deficient virus prepared as described above is attached to the yeast cell wall particle, a RNA protecting component, as described above, may also be attached to the particle.

In some therapeutic embodiments, it is beneficial for the vector to stably integrate into the target cell chromosome. For example, one mode for achieving stable integration is through the use of an adenovirus hybrid. Such an adenovirus hybrid involves, for example, an adenoviral vector carrying retrovirus 5′ and 3′ long terminal repeat (LTR) sequences flanking the DNA component encoding a therapeutic or antigenic nucleic acid or protein and a retrovirus integrase gene (see Zheng, et al. Nature Biotechnology, 18:176-180, 2000). In other embodiments, transient expression is preferred and cytoplasmic viruses, like Sindbis virus, can be employed. In such cases, where no lysosome evading component is naturally present on the virus, one is added. In the case of Sindbis or other such viruses, it can be engineered to express all or part of the adenovirus penton protein for this purpose, for example.

### Method for Attaching the Components to the Particle

Attachment of the components discussed above to the vector particle conjugate is accomplished by any means. As set out above, the various “components” include a nucleic acid, a lysosome evading component, which may both be present in a virus. Preferred methods for attachment include antibody attachment, biotin-(strept)avidin interaction and chemical crosslinking. Vector particle conjugates may be prepared with chemically attached antibodies, (strept)avidin or other selective attachment sites.

Antibody attachment can occur via any antibody interaction. Antibodies include, but are not limited to polyclonal antibodies, monoclonal antibodies (mAbs), humanized or chimeric antibodies, single chain antibodies including single chain Fv (scFv) fragments, Fab fragments, F(ab′)₂ fragments, fragments produced by a Fab expression library, anti-idiotypic (anti-Id) antibodies, epitope-binding fragments, and humanized forms of any of the above.

In general, techniques for preparing polyclonal and monoclonal antibodies as well as hybridomas capable of producing the desired antibody are well known in the art (Campbell, A. M., Monoclonal Antibody Technology: Laboratory Techniques in Biochemistry and Molecular Biology, Elsevier Science Publishers, Amsterdam, The Netherlands (1984); St. Groth et al., J. Immunol. Methods 35:1-21 (1980); Kohler and Milstein, Nature 256:495-497 (1975)), the trioma technique, the human B-cell hybridoma technique (Kozbor et al., Immunology Today 4:72 (1983); Cole et al., in Monoclonal Antibodies and Cancer Therapy, Alan R. Liss, Inc. (1985), pp. 77-96).

One example of antibody attachment encompassed by the present invention involves a single antibody which is chemically affixed to the digestible particle containing composition. The antibody is specific to the component to be attached to the particle. Alternatively, two antibodies can be used. In this case, one antibody, attached to the digestible particle is specific for a second antibody and the second antibody is specific to the component attached to the digestible particle. Thus, the component-specific antibody binds the component, and that antibody, in turn, is bound by the particle-bound antibody. For instance, a goat- or rabbit-anti-mouse antibody may be bound to the particle and a mouse monoclonal antibody used to bind the specific component.

In another example of antibody attachment, protein A or any similar molecule with an affinity for antibodies, is employed. In this example, the digestible particles are coated with protein A which binds to an antibody, which in turn is bound to the component being attached to the particle.

Attachment via biotin-(strept)avidin interaction may be accomplished, for instance, by attaching avidin to the digestible particle and attaching biotin to the component to be attached. Chemical crosslinking may be accomplished by conventional means known to the artisan.

Another attachment mechanism involves the nucleic acid serving as a multiple binding vehicle. Synthetic gripper protein nucleic acid (PNA) oligonucleotides are designed to specifically bind to different nucleic acid sequences. PNA is a polynucleic acid analog with a peptide backbone rather than a deoxyribosephosphate backbone. These can be attached directly to the digestible particle or derivatized for convenient attachment, thereby providing a sequence-specific means of attaching nucleic acid. Each gripper oligonucleotide can be derivatized or attached to different ligands or molecules and designed to bind different nucleic acid sequences. It is believed that the PNA interacts with the DNA via Hoogsteen base pairing interactions and that a stable PNA-DNA-PNA triplex clamp is formed (Zelphati, et al. BioTechniques, 28:304-316, 2000).

Thus, in one embodiment, one gripper is employed to bind the nucleic acid component to the particle and another is used to bind the lysosome evading component to the nucleic acid component. Many such iterations are possible. For example, a “gripper” comprising biotin can be sequence specifically bound at one site to the nucleic acid. Attachment to a particle coated with avidin occurs via biotin-avidin interaction. At another site on the nucleic acid, another “gripper” with a lysosome/phagasome evading component can be sequence specifically bound. Optionally, a “gripper” with a DNA protecting component can be sequence specifically bound to the nucleic acid at yet another site. Exemplary gripper oligonucleotides have been previously described.

In the case of attaching viruses to digestible particle, this can also be accomplished by engineering the virus to express certain proteins on its surface. For instance, the HIV env protein might be replaced with the adenovirus penton protein, or a portion thereof. The recombinant virus then could be attached via an anti-penton antibody, with attachment to the particle mediated, for example, by another antibody or protein A. In this embodiment, the penton protein also would serve as a lysosome evading component.

### Formulation

The digestible particle containing composition may be formulated for parenteral administration by, for example, local application (direct injection or microsurgery techniques), intramuscular or subcutaneous injection. Alternatively, the monocytic cells may be transfected ex vivo with the digestible particle containing composition and then reinfused intravenously into the patient.

Formulations for injection may be presented in unit dosage form, e.g., in ampules or in multi-dose containers, optionally with an added preservative. The compositions may take such forms as suspensions, solutions or emulsions in oily or aqueous vehicles, and may contain formulatory agents such as suspending, stabilizing and/or dispersing agents. The digestible particle containing composition may also be formulated using a pharmaceutically acceptable excipient. Such excipients are well known in the art, but typically will be a physiologically tolerable aqueous solution. Physiologically tolerable solutions are those which are essentially non-toxic. Preferred excipients will either be inert or enhancing.

### Therapeutic Methods

The digestible particle containing compositions of the present invention attract monocytes. It is, therefore, useful for any application involving selectively introducing a nucleic acid component into a monocyte cell, including gene vaccination, cancer treatment and gene therapy. Typical methods entail contacting a monocytic cell with a digestible particle containing composition.

The digestible particle containing compositions come into contact with monocytic cells either in vivo or in vitro. Hence, both in vivo and ex vivo methods are contemplated. As for in vivo methods, the digestible particle containing composition is generally administered parenterally, usually intravenously, intramuscularly, subcutaneously or intradermally. It may be administered, e.g., by bolus injection or continuous infusion. In ex vivo methods, monocytic cells are contacted outside the body and the contacted cells are then administered to the patient. The cells also are administered parenterally, typically via infusion.

In the field of vaccination, monocytic cells, including dendritic cells and macrophages, are considered “professional” antigen presenting cells (APCs) and, thus, are the ideal site for expression of a genetic vaccine. It is well known that expression of an antigen within an APC is vastly more effective in generating a strong cellular immune response than expression of this same antigen within any other cell type. Therefore, the ability of the digestible particle containing compositions of the instant invention to direct the expression of a vaccinating antigen to “professional” antigen presenting cells (monocyte cells) dramatically enhances the efficacy of a genetic vaccine.

The present invention offers a substantial improvement over the prior art vaccines because of the better tolerance macrophages have of phagocytized digestible particles compared with the magnetic beads. Also, the digestible particle containing compositions may be injected directly into the patient and target cells of monocytic origin, like macrophages and dendritic cells. Thus, the digestible particle containing compositions may be administered just like a conventional vaccine, which substantially reduces cost because of the lower level of skill required. Moreover, it is contemplated that altering the route of administration can alter the monocytic cells targeted.

Typical methods for gene vaccination involving compositions of the present invention include administering, generally subcutaneously or intravenously, to a patient a digestible particle containing composition. Alternatively, monocytic cells may be contacted with the digestible particle containing compositions described herein ex vivo and then the cells themselves parenterally administered to the patient. In addition, this ex vivo method may be modified by isolated T lymphocytes, using the contacted monocytic cells to ex vivo generate antigen-specific cytotoxic T cells, which then may be administered to the patient. The skilled artisan will be familiar with such strategies.

In addition to improved vaccination strategies, targeting gene expression to the monocytic cell lineage using the digestible particle containing compositions of the instant invention is effective for cancer treatment. One type of cancer treatment encompassed by the instant invention involves targeting a therapeutic gene to a tumor. It is known that as tumors, primary tumors and metastases alike, grow beyond a few millimeters in diameter and become deficient in oxygen, they secrete signal proteins to elicit several required events for the tumor's survival. These events include the secretion of signals which induce angiogenesis. As a part of the mechanism of angiogenic induction, hypoxic tumors secrete a signaling chemokine protein with the function of attracting monocytes to the tumor. Monocytes attracted to the sites of growing tumors then become macrophages and assist in the induction of tumor angiogenesis. Therefore, an effective method of therapeutic gene tumor targeting involves administering an effective amount of a digestible particle containing composition comprising an anti-tumor gene to a cancer patient, either directly or via an ex vivo contacted monocytic cell. The monocyte cells containing the phagocytized digestible particle containing composition should be attracted to the sites of tumor development and deliver the therapeutic tumor gene selectively to the tumor. In one embodiment, the therapeutic gene is under the control of a hypoxia induced promoter.

In another embodiment, the anti-tumor gene encodes an anti-angiogenic factor, like endostatin or angiostatin. Such a treatment is envisioned to be highly effective because it utilizes the monocytes as a delivery vehicle for anti-antiogenic factors.

In yet another embodiment, the anti-tumor gene may be an immunomodulator or an anti-inflammatory factor. Immunomodulators like IL-2 and IL-12 are envisioned. Moreover, anti-inflammatory factors not only would be useful in treating tumors, but also in treating chronic inflammatory disorders like arthritis. The anti-inflammatory effects of the invention, like the anti-tumor effects, rely on the ability of monocytic cells to home to the particular tissue. It is well known that monocytes are attracted to the sites of inflammatory response, like those in arthritis. Other exemplary immunomodulators and anti-inflammatories include GM-CSF and soluble TNF-alpha receptor.

Another use of the inventive compositions described herein is in conventional gene therapy. Typical methods for gene therapy involving the present invention include administering to a patient a composition which contains a yeast cell wall particle attached to a nucleic acid encoding a protein, or a monocytic cell (such as a macrophage or dendritic cell) containing that composition, to a patient.

The following non-limiting examples are given by way of illustration only and are not to be considered limitations of this invention. There are many apparent variations within the scope of this invention.

### EXAMPLE 1

This example demonstrates transfection of macrophages with Adenovirus-vectors coupled to particulate carriers.

### 1. Coupling of Adenoviral Vectors to Streptavidin-Magnetic Beads

Adenovirus (Ad) particles, suspended in PBS were biotinylated with Sulfo-NHS-LC-Biotin and added to Streptavidin-conjugated magnetic beads (MB) at a ratio of approximately 10 Ad particles/bead for 2 hours. Ad-MB conjugates were extensively washed with PBS and stored at 4° C. for further use.

### 2. Coupling of Adenoviral Vectors to Zymosan

2.1. Derivatization of Zymosan for Conjugation

Zymosan carbohydrate groups were mildly oxidized by sodium meta periodate, followed by addition of adipic acid dihydrazide (ADH) to introduce amino groups. The resulting conjugate was stabilized by addition of sodium cyanoborohydride. ADH-modified Zymosan was further reacted with SPDP (N-succinimidyl 3-(2-pyridyldithio) propionate) to introduce approximately 10⁶ reactive protected sulfhydryl groups per particle and extensively washed with PBS and stored at 4° C. for further use.

2.2. Modification of Adenoviral Vectors for Conjugation to Zymosan

Ad particles were reacted with SPDP for 2 hours to introduce protected sulfhydryl groups and subsequently purified from reaction by-products by spin-chromatography on Zeba spin columns (Pierce).

2.3. Coupling of Adenoviral Vectors to Modified Zymosan

Protected sulfhydryl groups in modified Zymosan described in step 2.1 were mildly reduced by Dithiothreitol for 30 minutes and washed extensively to remove the residual reagent. Subsequently, approximately 10 Ad particles, modified as described in 2.2, were added per Zymosan particle and allowed to react overnight to form stable disulfide bonds between Zymosan and the Ad particles. Ad-Z(ymosan) conjugates were washed extensively with PBS and stored at 4° C. for further use.

3. Transfection of Mouse Peritoneal Macrophages with Particulate Ad-Vectors

Thioglycollate elicited mouse peritoneal macrophages were seeded into 96-well culture plates in serum-free medium at a density of 10⁵ cells/well, allowed to adhere for 3 hours and subsequently washed to remove nonadherent cells. Thereafter, Ad-MB or Ad-Z conjugates were added at a ratio of approximately 4 particles per macrophage (equivalent to about 40 Ad particles) and incubated for 24 hours at 37° C. After this period, transfection efficiency was monitored by fluorescence microscopy for expression of the GFP transgene introduced by the Ad-vector.

### EXAMPLE 2

This example demonstrates stimulated secretion of macrophage antitumor activity by adenovirus-mediated gene transfer.

Thioglycollate elicted mouse peritoneal macrophages were transfected with Ad-Z(ymosan)-vectors at a ratio of approximately 4 Zymosan particles (equivalent to about 40 Ad-particles) per macrophage for 48 h. Thereafter, culture medium was collected, cleared by filtration (0.22 μm), concentrated 50-fold by ultrafiltration (cut-off 10 kDa), and dialyzed against HEPES-buffered saline (HBS). Serial dilutions of concentrated macrophage culture supernatants were incubated with YAC-1 mouse lymphoma cells for 40 h. Thereafter, viable tumor cells were stained with MTT and relative cytotoxicity of samples was determined photometrically with respect to controls incubated with HBS. Cytotoxicity is displayed as U/ml, with 1 U/ml defined as the concentration resulting in 50% cytotoxicity. Culture supernatants of unstimulated and untransfected macrophages or macrophages stimulated with bacterial lipopolysaccharide (LPS) served as a positive or negative controls respectively. The results show enhanced secretion of tumor cytotoxic activity after transfection with a mixture of two different RNAi constructs for IκB (Z-Ad406 and Z-Ad407) whereas control Ad-Z-vectors lacking the RNAi constructs (Z-AdGFP) caused only modestly enhanced secretion of macrophage tumor cytotoxic activity.

What is claimed is:

1. A method for directed entry into a monocytic cell, comprising contacting the monocyte with a composition comprising (i) a nucleic acid component, (ii) a lysosome evading component, and (iii) digestible particle that can be phagocytosed.

2. The method of claim 1, wherein the digestible particle that can be phagocytosed is from a natural source.

3. The method of claim 2, wherein the digestible particle that can be phagocytosed is of microbial origin.

4. The method of claim 2, wherein the digestible particle that can be phagocytosed is a yeast cell wall particle.

5. The method of claim 1, wherein the monocytic cell is a macrophage.

6. The method of claim 1, wherein the monocytic cell is a dendritic cell.

7. The method of claim 1, wherein the nucleic acid is selected from the group consisting of DNA and RNA.

8. The method of claim 1, wherein the nucleic acid is encoded in an expression vector.

9. The method of claim 5, wherein the expression vector contains a nuclear promoter.

10. The method of claim 6, wherein the promoter is a hypoxia induced promoter.

11. The method of claim 1, wherein the nucleic acid encodes a protein or an RNAi construct.

12. The method of claim 8, wherein the protein is an antigen.

13. The method of claim 1, wherein the lysosome evading component is a non-infectious virus or non-infectious component of a virus.

14. The method of claim 10, wherein the virus is adenovirus.

15. The method of claim 10, wherein the virus is non-replicative.

16. The method of claim 10, wherein the lysosome evading component is a biomimetic polymer.

17. The method of claim 1, wherein the digestible particle has a size between about 0.05 micron to about 5.0 μm.

18. The method of claim 1, wherein the digestible particle has a size between about 1.0 micron to about 2.5 μm.

19. The method of claim 4, wherein they yeast cell wall particle is zymosan.

20. The method of claim 1, further comprising a nucleic acid protecting component.

21. The method of claim 17, wherein the component is selected from the group consisting of protamine, polyarginine, polylysine, histone, histone-like proteins, synthetic polycationic polymers and a core particle of a retrovirus with the appropriate packaging sequence included in the RNA sequence.

22. The method of claim 1, wherein said nucleic acid and the lysosome evading component are attached to the particle by antibody attachment.

23. The method of claim 1, wherein the nucleic acid and said lysosome evading component are attached to the particle by interaction between (strept)avidin and biotin.

24. The method of claim 1, further comprising a multiple binding vehicle that binds the nucleic acid.

25. The method of claim 1, wherein the lysosome evading component is the adenovirus penton protein.

26. The method of claim 1, wherein the composition is a pharmaceutical composition that comprises a pharmaceutically suitable excipient.

27. The method of claim 23, wherein the nucleic acid encodes an antigen.

28. A composition comprising (i) a nucleic acid component, (ii) a lysosome evading component, and (iii) a digestible particle that can be phagocytosed.

29. The composition of claim 25, wherein the digestible particle that can be phagocytosed is from a natural source.

30. The composition of claim 25, wherein the digestible particle that can be phagocytosed is of microbial origin.

31. The composition of claim 25, wherein the digestible particle that can be phagocytosed is a yeast cell wall particle.

32. The composition of claim 25, wherein the monocytic cell is a macrophage.

33. The composition of claim 25, wherein the monocytic cell is a dendritic cell.

34. The composition of claim 25, wherein the nucleic acid is selected from the group consisting of DNA and RNA.

35. The composition of claim 25, wherein the nucleic acid is encoded in an expression vector.

36. The composition of claim 29, wherein the expression vector contains a nuclear promoter.

37. The composition of claim 30, wherein the promoter is a hypoxia induced promoter.

38. The composition of claim 25, wherein the nucleic acid encodes a protein or an RNAi construct.

39. The composition of claim 32, wherein the protein is an antigen.

40. The composition of claim 25, wherein the lysosome evading component is a non-infectious virus or non-infectious component of a virus.

41. The composition of claim 34, wherein the virus is adenovirus.

42. The composition of claim 34, wherein the virus is non-replicative.

43. The composition of claim 34, wherein the lysosome evading component is a biomimetic polymer.

44. The composition of claim 25, wherein the digestible particle has a size between about 0.05 micron to about 5.0 μm.

45. The composition of claim 25, wherein the digestible particle has a size between about 1.0 micron to about 2.5 μm.

46. The composition of claim 31, wherein the yeast cell wall particle is zymosan.

47. The composition of claim 25, further comprising a nucleic acid protecting component.

48. The composition of claim 41, wherein the component is selected from the group consisting of protamine, polyarginine, polylysine, histone, histone-like proteins, synthetic polycationic polymers and a core particle of a retrovirus with the appropriate packaging sequence included in the RNA sequence.

49. The method of claim 25, wherein said nucleic acid and the lysosome evading component are attached to the particle by antibody attachment.

50. The method of claim 25, wherein the nucleic acid and said lysosome evading component are attached to the particle by interaction between (strept)avidin and biotin.

51. The method of claim 25, further comprising a multiple binding vehicle that binds the nucleic acid.

52. The method of claim 25, wherein the lysosome evading component is the adenovirus penton protein.

53. The method of claim 25, wherein the composition is a pharmaceutical composition that comprises a pharmaceutically suitable excipient.
